(19) United States
(12) Patent Application Publication
(10) Pub. No.: US 2016/0145591 A1
(43) Pub. Date: May 26, 2016

(54) BACTERIAL LYSINS AND USES THEREOF

(71) Applicant: UNIVERSITE DE LAUSANNE, Lausanne (CH)

(72) Inventors: Grégory RESCH, Evian (FR); Frank OECHSLIN, Le Mont (CH); Philippe MOREILLON, Lausanne (CH)

(21) Appl. No.: 14/900,530

(22) PCT Filed: Jul. 15, 2014
(86) PCT No.: PCT/IB2014/063103

§ 371 Date: Dec. 21, 2015

(30) Foreign Application Priority Data
Jul. 16, 2013 (EP) 13176730.3

(51) Int. Cl. C12N 9/24; C12Q 1/40; A01N 63/00; A61K 38/47

(52) U.S. Cl. CPC C12N 9/2402; A61K 38/47; C12Q 1/40; A01N 63/00; G01N 2333/315; G01N 2333/924; C12Y 302/01017

(57) ABSTRACT

The present invention relates to substances and compositions thereof useful in the prevention and/or treatment of bacterial infections and disorders, in particular bacteremia caused by group B Streptococcus. In another aspect, the invention relates to substances and compositions thereof useful for decontaminating biological or inanimate materials which could be contaminated by Gram-positive bacteria. In a further aspect, the invention relates to substances and compositions thereof useful for detecting the presence of certain Gram-positive bacteria in biological or inanimate materials.

### FIELD OF THE INVENTION

The present invention provides methods and compounds for treating, decontaminating, or detecting, bacterial infections and disorders.

### BACKGROUND OF THE INVENTION

Streptococcus agalactiae or group B Streptococcus (GBS) is a common inhabitant of the gastrointestinal and genital tracts with an asymptomatic carriage of 9% to 30% in adults. GBS strains are classified in 10 different serotypes in function of their capsular antigen, serotypes Ia, II, III, and V being the most common infectious one. Currently, GBS is the leading cause of severe and invasive neonatal infections. Moreover, it causes high morbidity in pregnant women, elderly people and adults with medical underlying conditions like diabetes, cancer, HIV infection, and chronic infections. In neonates, GBS infections are classified either as early or late onset diseases. The early onset disease occurs within a few days after birth and is the result of direct vertical transmission during delivery. In this setting, neonates become infected during the passage through the birth canal or by contact with amniotic fluids contaminated by the ascending spread of the bacteria from the vagina after the rupture of the amniotic membrane. Early onset disease results in 80% of the overall diseases in neonates and occurs at a frequency of 0.5-3 per 1000 live births in developed countries. In 74% of the cases, GBS infections leads to bacteremia, but also meningitis (14%) or pneumonia (12%), with a mortality rate ranging from 5% to 20% (Money et al. 2004, J Obstet Gynaecol Can. 26(9): 826-40). Late onset disease can occur up to the third month after delivery and is unrelated to vaginal colonisation. In this type of GBS-associated disease, bacteremia is also the major outcome (25% of the cases) followed by meningitis which leads to permanent neurological sequels with mental retardation, blindness or hearing loss in 20% of the case (Shet et al. 2004, Indian J Med Res. 120(3): 141-50). Currently, intra-partum antibiotics prophylaxis is the approved approach by Centers for Disease Control and Prevention (CDC). It consists in β-lactam antibiotics administration 4 h before delivery to prevent, with an efficacy of 60%, the vertical transmission of GBS and subsequent early onset disease (Rutledge et al. 2010, Morbidity Mortality Weekly Report 59). In contrast, no strategy is currently available to prevent late onset diseases, which incidence is now over passing the early onset diseases.

Recently, concerns have emerged regarding GBS isolates with increased penicillin and ampicillin minimum inhibitory concentrations (MIC) or penicillin G and ceftriaxone un-susceptible isolates. In the United States and Canada, resistant strains to erythromycin and clindamycin have increased to reach a prevalence of 3%-21% and 5%-29%, respectively (Castor et al., 2008, Infect. Dis. Obstet. Gynecol., 727505). This is of high concern regarding the fact that both drugs are second line therapeutic agents for penicillin allergic patients. Furthermore, the fatality rate of early onset diseases raised from 6.5% in 1996 to 16.5% in 2005 in Finland, a phenomenon suggested to be related to the increase of resistance to erythromycin and clindamycin (Bergseng et al., 2009, Clin Microbiol Infect., 15(12):1182-5).

To overcome this serious and general problem of antibiotic resistance, different approaches are currently investigated. One new class of antibacterial agents, i.e. bacteriophages derived peptidoglycan hydrolases called lysins or enzybiotics, is getting increasing attention due to their therapeutic successes in a wide range of animal models (Fischetti, 2008, Curr. Opin. Microbiol., 11(5):393-400). The majority of lysins from phages infecting Gram-positive pathogens described so far have a modular structure with both a catalytic domain (CD) and a cell wall binding domain (CBD) that are linked together by a short linker (Hermoso et al., 2003, Structure. 11(10):1239-49). These enzymes are categorized in four main groups (amidase, muramidase or lysozyme, endopeptidase, and glucosaminidase) depending on the peptidoglycan cleavage sites targeted by their catalytic domains.

Currently, only four lysins with antibacterial activity against GBS have been described, among which B30 lysin and its very similar homolog PlyGBS, LambdaSa1, and LambdaSa2.

The B30 enzyme, isolated from the GBS bacteriophage B30, carries two different catalytic subunits, i.e. a cysteine histidine-dependent amidohydrolase/peptidase (CHAP) domain with an endopeptidase activity and an acetylmuramidase domain with a glycosidase activity (Pritchard et al., 2004, Microbiology, 150(Pt 7):2079-87). Single point mutations in the conserved cysteine or histidine residues of the CHAP domain demonstrated that it is responsible for nearly all the lytic activity of the enzyme (Donovan et al., 2006, Appl Environ. Microbiol., 72(7):5108-12). In addition, the lysin also contains a C-terminal SH3b CBD (Baker et al., 2006, Appl. Environ. Microbiol., 72(10): 6825-8) and its truncation revealed that it is required for the glycosidase activity (Donovan et al., 2006, supra). B30 lysin from Group B streptococcal bacteriophage B30 has been found to be able of lysing several β-hemolytic streptococci in vitro including groups A, B, C, E and G streptococci. However, the rate of lysis by B30 lysin on Group B streptocci was low and its optimum pH ranging between 5.5 and 6 (Pritchard et al., 2004, supra).

Another well studied recombinant streptococcal lysin, PlyGBS, isolated from the GBS phage NCTC 11261 and having 99% homology with B30 lysin, is active against group A, B, C, G and L streptococci. It has been developed as a prophylactic for Group B streptococcal vaginal colonization in pregnant women before infant delivery and also for use as a decontaminant to eliminate Group B streptococcal from new-borns (Cheng et al., 2005, Antimicrobial Agents and Chemotherapy, 49(1): 111-117). The optimum pH of PlyGBS (about 5) is within the range normally found in the human vaginal tract. PlyGBS mutants have been produced which have up to 28-fold better activity against GBS than the wild-type enzyme, with an optimal pH unchanged. For instance, a truncated version of the PlyGBS containing only the CHAP domain with an extra 13 amino sequence at the C-terminal end showed superior lytic activity (Cheng et al., 2007, Appl. Microbiol. Biotechnol., 74(6):1284-91).

LambdaSa1 and LambdaSa2 correspond to two prophage lysins present in the GBS strain 2603 V/R showing endopeptidase activity against GBS, S. aureus, and S. pneumoniae cell walls (Pritchard et al., 2007, Appl. Environ. Microbiol., 73(22): 7150-4). Neither data regarding in vitro killing assays nor in vivo therapeutic trials in animal appears to have been published with LambdaSa1 or LambdaSa2 yet.

Although some bacteriophage lysins are already available, there remains a need for alternative lysins exhibiting different properties such as regarding the specifically targeted group of Gram-positive bacteria, level of activity, optimum pH, in order to provide means for treating a broad range of infections caused by different bacteria possibly affecting different tissues or allowing treatment at different stages of the infection or disease.

### SUMMARY OF THE INVENTION

The present invention is based on the unexpected properties and structure of a bacterial lysin isolated from Streptococcus dysgalactiae subsp. equisimilis (SDSE) strain SK1249 (Vandamme et al, 1996, Int J Syst Bacteriol 46:774-781, 1996, CCUG 36637), which make it particularly suitable for various uses and methods for treating, decontaminating, or detecting, bacterial infections and disorders, in particular in relation with group B Streptococcus (GBS).

A first aspect of the invention relates to an antibacterial composition comprising a polypeptide comprising the amino acid sequence SEQ ID NO: 1, or any variant thereof having at least 80% identity with SEQ ID NO: 1, or any fragment thereof, wherein said polypeptide has a killing activity against at least one Streptococcus agalactiae bacteria (GBS) at a pH comprised between about 7 and 8.5, at 37° C.

A second aspect of the invention relates to a pharmaceutical composition comprising a polypeptide as described above and a pharmaceutically acceptable carrier.

A third aspect of the invention relates to a polypeptide as described above, for use in the prevention and/or treatment of bacterial infections and disorders.

A fourth aspect of the invention concerns the use of a polypeptide as described herein, in the manufacture of a medicament for preventing and/or treating bacterial infections and disorders.

A fifth aspect of the invention provides a method for preventing and/or treating bacterial infections and disorders comprising administering, in a subject in need thereof, a therapeutically effective amount of a polypeptide or a composition thereof as described herein.

A sixth aspect of the invention concerns a method for decontaminating a biological material or an inanimate material or surface, comprising contacting said material, surface or medium with an effective amount of an antibacterial composition as described herewith.

In a seventh aspect of the invention, is provided a kit comprising a polypeptide as described herewith and instructions of use.

An eighth aspect of the invention relates to a method for detecting the presence of at least one streptococci bacteria, in or on a biological material or inanimate material or surface, comprising:
a) contacting said biological material or inanimate material or surface with an effective amount of a composition comprising a polypeptide as described herewith, covalently linked to a reporter protein or a radioactive, fluorescent, colorimetric, or chemiluminescent molecule, and
b) detecting a signal emitted by the reporter protein or radioactive, fluorescent, colorimetric, or chemiluminescent molecule, in or on said biological material or inanimate material or surface;
wherein the detection of a signal in step b) indicates the presence of said streptococci bacteria.

A ninth aspect of the invention concerns an isolated polypeptide as described herewith, for use as a medicament.

A tenth aspect of the invention concerns a composition comprising a polypeptide comprising or consisting of amino acid sequence SEQ ID NO:10, or a variant thereof capable of binding to the cell wall of Streptococcus agalactiae bacteria, covalently linked to a reporter protein or a radioactive, fluorescent, colorimetric, or chemiluminescent molecule.

Other features and advantages of the invention will be apparent from the following detailed description.

### DESCRIPTION OF THE FIGURES

FIG. 1. Schematic representation of different lysin polypeptides (LytN, PlySK1249, B30 Lysin and PlyGBS). Percentage of amino acid sequences identity is represented between two considered domains.

FIG. 2. In vitro activity of PlySK1249 against Streptococcus dysgalactiae subsp. equisimilis (SDSE) strain SK1249 and B Streptococcus (GBS) clinical strain 17-2167 measured as described in Example 2: by variations in Optical Density at 600 nm versus time after PlySK1249 addition (T) (Filled curve) by a time-kill assay with SDSE strain SK1249 (filled diamonds) and its respective control (clear diamonds) and by a time-kill assay with GBS clinical strain 17-2167 (filled circles) and its respective control (open circles). All experiments but controls were done in presence of 3.3 U/ml of PlySK1249. Each dot represents the mean of three different experiments.

FIG. 3. Activity of PlySK1249 in function of the pH measured as described in Example 3 by variations in Optical Density at 600 nm. All experiments were done in turbidity assays in presence of 3.3 U/ml of purified PlySK1249 and SDSE strain SK1249 harvested in mid-exponential growth phase (i.e. OD₆₀₀ₙₘ about 0.5). Bacterial cells were washed and resuspended in lysis buffer and adjusted to OD₆₀₀ₙₘ about 0.5 before addition of PlySK1249. Each dot represents the mean of three independent experiments.

FIG. 4. (A) Growth curve of SDSE strain SK1249 as measured by variations in Optical Density at 600 nm versus time (T) in aerobic conditions in brain heart infusion (BHI) at 37° C. and 250 r.p.m. (B) PlySK1249 activity in function of bacterial growth phase as described in Example 3 as measured by variations in Optical Density at 600 nm versus time after PlySK1249 addition (T). All experiments were done in turbidity assays by the addition of 3.3 U/ml of purified PlySK1249 to suspensions of SDSE strain SK1249 harvested in different phases of growth. Cells were washed and adjusted to OD₆₀₀ₙₘ about 0.5 in lysis buffer before addition of PlySK1249. Each dot represents the mean of three independent experiments.

FIG. 5. Activity spectrum of PlySK1249 on different bacterial species as measured by a decrease in Optical Density at 600 nm as described in Example 5. All experiments were done in turbidity assays in presence of 3.3 U/ml of purified PlySK1249 and bacterial cells harvested in mid-exponential growth phase (i.e. OD₆₀₀ₙₘ about 0.5), previously washed and adjusted to OD₆₀₀ₙₘ about 0.5 in lysis buffer. Each bar represents the mean of three independent experiments.

FIG. 6. Therapeutic effect of PlySK1249 in a mouse model of GBS-induced bacteremia as measured by the percentage of survival (S %) versus time (T) after bacterial challenge by i.p. injection of 10⁶ CFU of the GBS clinical strain 17-2167 as described in Example 5 (A) Single bolus i.p. injection. of 22.5 mg/kg PlySK1249 (closed circles). (B) Three bolus i.p. injections of 45 mg/kg (black arrows), i.e. a total of 135 mg/kg over the first 24 h, of PlySK1249 (closed circles). Open circles: control batch receiving lysin buffer only. Black arrows: time of injection after infection.

### DETAILED DESCRIPTION OF THE INVENTION

The terms “lysins”, “lysin polypeptides”, “bacterial lysins”, “enzybiotic”, or “phage lytic enzymes”, refer to phage-encoded peptidoglycan hydrolases which, when applied exogenously (as purified recombinant proteins for instance) to Gram-positive bacteria bring about rapid lysis and cell death of the bacterial cell as no membrane is present to inhibit their access to the cell wall. In general, lysins selectively target specific pathogenic bacteria without affecting surrounding commensal microflora, and have been reported to have a narrow host range similar to that of their phage rendering them, generally, either species or genus specific.

The terms “peptide”, “polypeptide”, “protein” and variations of these terms refer to peptide, oligopeptide, oligomer or protein including fusion protein, respectively, comprising at least two amino acids joined to each other by a normal or modified peptide bond, such as in the cases of the isosteric peptides, for example. These terms also include herewith “peptidomimetics” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. A peptide or polypeptide can be composed of amino acids other than the 20 amino acids defined by the genetic code. It can be composed of L-amino acids and/or D-amino acids. A peptide or polypeptide can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain or even at the carboxy- or amino-terminal ends. A peptide or polypeptide can be branched following an ubiquitination or be cyclic with or without branching. This type of modification can be the result of natural or synthetic post-translational processes that are well known to a person skilled in the art. For example, peptide or polypeptide modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature (Proteins Structure and Molecular Properties (1993) 2nd Ed., T E. Creighton, New York; Post-translational Covalent Modifications of Proteins (1983) B. C. Johnson, Ed., Academic Press, New York; Seifter et al. (1990) Analysis for protein modifications and nonprotein cofactors, Meth. Enzymol. 182: 626-646 and Rattan et al., (1992) Protein Synthesis: Post-translational Modifications and Aging, Ann NY Acad Sci, 663: 48-62).

The term “bacteremia” refers herewith to the presence of viable bacteria in the blood stream. Bacteria can enter the bloodstream as a severe complication of infections like pneumonia or meningitis, during surgery (especially when involving mucous membranes such as the gastrointestinal tract), or due to catheters and other foreign bodies entering the arteries or veins (including intravenous drug abuse). Bacteremia can have several consequences. The immune response to the bacteria can cause sepsis and septic shock, which has a relatively high mortality rate. Bacteria can also use the blood to spread to other parts of the body, causing infections away from the original site of infection, such as in endocarditis or osteomyelitis.

As used herewith “bacterial infections and disorders” refer to infections and disorders caused by Gram-positive bacteria, in particular infections and disorders caused by at least one streptococci bacteria selected from the group consisting of Streptococcus agalactiae (GBS), Streptococcus dysgalactiae (GCS), Streptococcus pyogenes (GAS), Streptococcus uberis, Streptococcus suis and Streptococcus gordonii”. Bacterial infections and disorders include herewith bacteremia, meningitis, pneumonia, streptococcal toxic shock syndrome, necrotizing fasciitis, septicemia, endocarditis, deafness, mastitis.

As defined herewith the terms “killing activity” of a polypeptide against a particular bacteria represents a reduction in the number of viable bacteria cells caused by the lysing activity of said polypeptide. The killing activity of the polypeptide against said bacteria can be complete meaning that 100% of the bacterial cells have been lysed or partial meaning that at least about 20%, at least about 30%, at least about 40%, at least about 50%, or at least about 60% of the bacterial cells have been lysed. Killing activity can be determined by measuring a decrease in optical density at 600 nm of a bacterial cell suspension and/or a decrease in Colony Forming Units (CFU) per millilitre of a bacterial cell suspension after exposure to the polypeptide to be tested.

As defined herewith the terms “binding capacity” of a polypeptide to the cell wall of a particular bacteria refers to the ability of said polypeptide to specifically interact and adhere to the cell wall of said bacteria. The binding capacity of a polypeptide to the cell wall of a bacteria can be determined by Surface Plasmon Resonance (Stahelin R V, 2013, Mol. Biol. Cell, 24(7):883-6).

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it for example in neonates resulting from direct vertical transmission from the infected mother during delivery; (b) inhibiting the disease, i.e., arresting its development; or relieving the disease, i.e., causing regression of the disease and/or its symptoms or conditions such as improvement or remediation of damage. In particular, treatment of bacterial infections comprises preventing, decreasing or even eradicating the infection, for instance by killing the bacteria and, thus, controlling, reducing or inhibiting bacterial proliferation as well as reducing the number of viable bacterial cells.

The term “subject” as used herein refers to mammals. For examples, mammals contemplated by the present invention include human, primates, domesticated animals such as cattle, sheep, pigs, horses, laboratory rodents and the like.

The term “effective amount” as used herein refers to an amount of at least one polypeptide according to the invention, composition or pharmaceutical formulation thereof, that elicits the biological or medicinal response in a tissue, system, animal or human that is being sought. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active polypeptide sufficient to reduce the progression of the disease, notably to reduce or inhibit the disorder or infection and thereby elicit the response being sought (i.e. an “inhibition effective amount”).

The term “efficacy” of a treatment according to the invention can be measured based on changes in the course of disease in response to a use or a method according to the invention. The efficacy of prevention of infectious disease is ultimately assessed by epidemiological studies in human populations, which often correlates with titers of neutralizing antibodies in sera, and induction of multifunctional pathogen specific T cell responses. Preclinical assessment can include resistance to infection after challenge with infectious pathogen. Treatment of an infectious disease can be measured by inhibition of the pathogen's growth or elimination of the pathogen (and, thus, absence of detection of the pathogen), correlating with pathogen specific antibodies and/or T cell immune responses.

The term “biological material” refers to any material or sample that is obtained from a subject's body. This includes, for instance, samples of whole blood, serum, plasma, urine, sputum, saliva, vaginal swabs, or spinal fluids.

The term “inanimate material or surface” includes solutions, medium, devices, objects, floor, surface of a table.

The term “medium” includes water, air or food.

The term “pharmaceutical formulation” refers to preparations which are in such a form as to permit biological activity of the active ingredient(s) to be unequivocally effective and which contain no additional component which would be toxic to subjects to which the said formulation would be administered.

The term “pharmaceutically acceptable” refers to a carrier comprised of a material that is not biologically or otherwise undesirable.

The term “carrier” refers to any components present in a pharmaceutical formulation other than the active agent and thus includes diluents, binders, lubricants, disintegrants, fillers, coloring agents, wetting or emulsifying agents, pH buffering agents, preservatives and the like.

Polypeptides According to the Invention

In a first aspect of the invention is provided a polypeptide isolated from Streptococcus dysgalactiae subsp. equisimilis (SD SE) strain SK1249 that has an antibacterial activity against Streptococcus agalactiae (Group B streptococcus). The optimum pH at which the polypeptide according to the invention exhibits an antibacterial activity is comprised between about 7 and 8.5.

In a particular embodiment, the polypeptide of the invention comprises the amino acid sequence SEQ ID NO: 1 (GenBank accession number EGL49245.1). It can be encoded by a gene of nucleotide sequence SEQ ID NO: 2.

In a further embodiment, the polypeptide of the invention includes any variant of the polypeptide having the amino acid sequence SEQ ID NO: 1, said variant having at least 80%, at least 90%, at least 95%, at least 96%, at least 97%, at least 98%, or at least 99% identity with SEQ ID NO: 1 and having a killing activity against at least one Streptococcus agalactiae bacteria (GBS) at a pH comprised between about 7 and 8.5, at 37° C.

The percentage of identity between two amino acid sequences or two nucleic acid sequences can be determined by visual inspection and/or mathematical calculation, or more easily by comparing sequence information using a computer program such as Clustal package version 1.83.

In a still further embodiment, the polypeptide of the invention comprises any fragment of the amino acid sequence SEQ ID NO: 1, or fragment of a variant thereof as defined above, provided said fragment has an antibacterial activity against Streptococcus agalactiae (Group B streptococcus), in particular at pH comprised between about 7 and 8.5, at 37° C.

In particular, said fragment can comprise the amino acid sequence SEQ ID NO: 9, or any variant thereof having at least 80%, at least 90%, at least 95%, at least 96%, at least 97%, at least 98%, or at least 99% identity with SEQ ID NO: 9, wherein said fragment or variant thereof has an antibacterial activity (e.g. a killing activity) against at least one Streptococcus agalactiae bacteria (GBS) at a pH comprised between about 7 and 8.5, at 37° C.

In a another embodiment, the polypeptide of the invention comprises any fragment of the amino acid sequence SEQ ID NO: 1, or variant thereof as defined above, provided said fragment is capable of binding to the cell wall of Streptococcus agalactiae (Group B streptococcus).

In particular, said fragment can comprise the amino acid sequence SEQ ID NO: 10, or any variant thereof having at least 80%, at least 90%, at least 95%, at least 96%, at least 97%, at least 98%, or at least 99% identity with SEQ ID NO: 10, wherein said fragment or variant thereof is capable of binding to the cell wall of Streptococcus agalactiae (Group B streptococcus).

According to one aspect of the invention, the isolated polypeptide according to the invention, variant thereof, or fragment thereof, comprises an amino acid sequence having at least one conservatively substituted amino acid from the native sequence, meaning that a given amino acid residue is replaced by a residue having similar physicochemical characteristics. Generally, substitutions for one or more amino acids present in the native amino acid sequence should be made conservatively. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity properties, are well known (Kyte and Doolittle, 1982, J. Mol. Biol. 157(1):105-132). For example, a “conservative amino acid substitution” may involve a substitution of a native amino acid residue with a non-native residue such that there is little or no effect on the polarity or charge of the amino acid residue at that position. Desired amino acid substitutions (whether conservative or non-conservative) can be determined by those skilled in the art at the time such substitutions are desired. Exemplary amino acid substitutions are presented in Table 1 below:

TABLE 1

| Original residues | Examples of substitutions |
|---|---|
| Ala (A) | Val, Leu, Ile |
| Arg (R) | Lys, Gln, Asn |
| Asn (N) | Gln |
| Asp (D) | Glu |
| Cys (C) | Ser, Ala |
| Gln (Q) | Asn |
| Glu (E) | Asp |
| Gly (G) | Pro, Ala |
| His (H) | Asn, Gln, Lys, Arg |
| Ile (I) | Leu, Val, Met, Ala, Phe, Norleucine |
| Leu (L) | Ile, Val, Met, Ala, Phe, Norleucine |
| Lys (K) | Arg, Gln, Asn |
| Met (M) | Leu, Ile, Phe |
| Phe (F) | Leu, Val, Ile, Ala, Tyr |
| Pro (P) | Ala, Gly |
| Ser (S) | Thr, Ala, Cys |
| Thr (T) | Ser |
| Trp (W) | Tyr, Phe |
| Tyr (Y) | Trp, Phe, Thr, Ser |
| Val (V) | Ile, Met, Leu, Phe, Ala, Norleucine |

The polypeptide of the invention may further exhibit a killing activity against at least one streptococci bacteria selected from the group consisting of Streptococcus dysgalactiae (GCS), Streptococcus pyogenes (GAS), Streptococcus uberis, Streptococcus suis, and Streptococcus gordonii.

In another embodiment, the polypeptide according to the invention is chemically modified, for instance by glycosylation including pegylation, methylation, and/or phosphorylation.

In a further embodiment, the polypeptide according to the invention is labelled so that it can be detected by standard techniques in the art.

In a particular embodiment, the polypeptide of the invention is labelled, for instance, with a radioactive, fluorescent, colorimetric, or chemiluminescent molecule, or fused to a reporter protein, according to standard methods in the art.

In the alternative aspect where the polypeptide according to the invention is fused to a reporter protein, one skilled in the art will understand that any reporter protein can be used including fluorescent proteins like Green Fluorescent Protein (GFP) and Red Fluorescent Protein (RFP), luminescent proteins like luciferase, as well as other reporter proteins like β-galactosidase, for instance.

In another particular embodiment, the polypeptide according to the invention is fused to another polypeptide like a different lysin polypeptide or a fragment thereof.

The killing activity of the polypeptide according to the invention on a particular microorganism may be determined by standard procedures in the field including those based on the determination of the Minimum Inhibitory Concentrations (MICs) of an antimicrobial agent defined as the lowest concentration of said antimicrobial agent that inhibits the visible growth of a microorganism after overnight incubation as described in Andrews, 2001, J Antimicrobial Chemotherapy, 48, Suppl. S1, 5-16) or in “Document M7 A7, Methods for dilution antimicrobial susceptibility tests for bacteria that grow aerobically; Approved standards, 7^{th} Edition, January 2006, vol. 26, No. 2” published by Clinical and Laboratory Standards Institute. Another suitable method for determining the killing activity of the polypeptide according to the invention is described in the example section of the present application and consists in determining the decrease of the Optical Density measured at 600 nm of a suspension of the bacteria the susceptibility of which is to be tested in an in vitro turbidity assay performed in presence of 3.3 U/ml of purified polypeptide according to the invention and of the bacteria cells harvested at an OD₆₀₀ₙₘ of about 0.5, previously washed and adjusted to OD₆₀₀ₙₘ in lysis buffer comprising 40 mM phosphate buffer, 200 mM NaCl, pH 7.4, at 37° C., after 15 min.

According to another embodiment, in an in vitro turbidity test as described herewith, a polypeptide according to the invention decreases the OD₆₀₀ₙₘ of a suspension of at least one strain of Streptococcus agalactiae bacteria (GBS) by more than 20%, more than 30%, more than 40%, more than 50%, or more than 60%. In particular, in an in vitro turbidity test as described herewith, with a concentration of 3.3 U/ml, a polypeptide according to the invention decreases the OD₆₀₀ₙₘ of a suspension of Streptococcus agalactiae FSL-S3 (GBS) by more than 60% and that of Streptococcus pyogenes ATCC 19615 (GAS) by more than 40%.

The polypeptide according to the invention can be produced by standard techniques of genetic engineering comprising the use of a recombinant vector comprising a polynucleotide encoding a polypeptide of amino acid sequence as described herewith. Numerous expression systems can be used including bacterial plasmids and derived vectors, transposons, yeast episomes, insertion elements, yeast chromosome elements, viruses such as baculovirus, papilloma viruses such as SV40, vaccinia viruses, adenoviruses, fox pox viruses, pseudorabies viruses, retroviruses, cosmid or phagemid derivatives. The nucleotide sequence can be inserted in the recombinant expression vector by methods well known to a person skilled in the art such as, for example, those that are described in MOLECULAR CLONING: A LABORATORY MANUAL, Sambrook et al., 4th Ed., Cold Spring Harbor Laboratory Press, Cold Spring Harbor, N. Y., 2001. The recombinant vector can include nucleotide sequences that control the regulation, the expression, the transcription, and/or the translation of the polynucleotide encoding the polypeptide, these sequences being selected according to the host cells that are used. The recombinant vector can further include nucleotide sequences such as those encoding His tags for facilitating the purification step.

Subsequently, such a recombinant vector is introduced in a host cell according to methods that are well known to a person skilled in the art, such as those described in BASIC METHODS IN MOLECULAR BIOLOGY, Davis et al., 2nd ed., McGraw-Hill Professional Publishing, 1995, and MOLECULAR CLONING: A LABORATORY MANUAL, supra, such as transfection by calcium phosphate, transfection by DEAE dextran, transfection, microinjection, transfection by cationic lipids, electroporation, transduction or infection.

The host cell can be, for example, bacterial cells such as E. coli, cells of fungi such as yeast cells and cells of Aspergillus, Streptomyces, insect cells, Chinese Hamster Ovary cells (CHO), C127 mouse cell line, BHK cell line of Syrian hamster cells, Human Embryonic Kidney 293 (HEK 293) cells. Preferably, the host cell is E. coli. Said host cells are then cultivated in appropriate conditions so as to produce the polypeptide described herewith, which can then further be purified from the culture medium or from the host cell lysate by any standard purification methods including, Immobilized-Metal Affinity Chromatography (IMAC) (Block et al. 2008, Protein Expr. Purif. 27, 244-254).

Compositions According to the Invention

In a further aspect of the invention are provided antibacterial compositions comprising a polypeptide according to the invention, in particular pharmaceutical compositions. In one embodiment is provided an antibacterial composition comprising a polypeptide comprising the amino acid sequence SEQ ID NO: 1, or any variant thereof having at least 80%, at least 90%, at least 95%, at least 96%, at least 97%, at least 98%, or at least 99% identity with SEQ ID NO: 1, or any fragment thereof, wherein said variant or fragment has an antibacterial activity (e.g. a killing activity) against at least one Streptococcus agalactiae bacteria (GBS) at a pH comprised between about 7 and 8.5, at 37° C.

In a particular embodiment is provided an antibacterial composition comprising a polypeptide comprising the amino acid sequence SEQ ID NO: 9, or any variant thereof having at least 80%, at least 90%, at least 95%, at least 96%, at least 97%, at least 98%, or at least 99% identity with SEQ ID NO: 9, wherein said variant has an antibacterial activity (e.g. a killing activity) against at least one Streptococcus agalactiae bacteria (GBS) at a pH comprised between about 7 and 8.5, at 37° C.

In a further embodiment is provided an antibacterial composition comprising a polypeptide comprising the amino acid sequence SEQ ID NO: 9, or any variant thereof having at least 80%, at least 90%, at least 95%, at least 96%, at least 97%, at least 98%, or at least 99% identity with SEQ ID NO: 9, wherein said variant has an antibacterial activity (e.g. a killing activity) against at least one Streptococcus agalactiae bacteria (GBS) at a pH comprised between about 7 and 8.5, at 37° C., and further comprising the amino acid sequence SEQ ID NO: 10, or any variant thereof having at least 80%, at least 90%, at least 95%, at least 96%, at least 97%, at least 98%, or at least 99% identity with SEQ ID NO: 10, wherein said variant is able to bind to the cell wall of at least one Streptococcus agalactiae bacteria (GBS).

In a further embodiment, is provided an antibacterial composition according to the invention wherein said polypeptide decreases the OD₆₀₀ₙₘ of a suspension of said at least one streptococci bacteria by more than 20% in an in vitro turbidity assay performed in presence of 3.3 U/ml of said polypeptide and streptococci bacterial cells harvested at an OD₆₀₀ₙₘ of about 0.5, previously washed and adjusted to OD₆₀₀ₙₘ in lysis buffer comprising 40 mM phosphate buffer, 200 mM NaCl, pH 7.4, at 37° C., after 15 min, wherein 1 U of polypeptide is defined as the amount of polypeptide that could decrease by half in 15 min and 37° C. the OD₆₀₀ₙₘ of a 300 μl suspension of streptococci bacterial cells that were harvested in the mid-logarithmic growth phase (i.e. about 0.5).

In a further embodiment, is provided an antibacterial composition according to the invention, wherein the polypeptide decreases the OD₆₀₀ₙₘ of a suspension of at least one strain of Streptococcus agalactiae bacteria (GBS) by more than 40%.

According to a further aspect, the antibacterial compositions of the invention are antibacterial pharmaceutical formulations.

According to another aspect of the invention, is provided a pharmaceutical composition comprising a polypeptide comprising the amino acid sequence SEQ ID NO: 1, or any variant thereof having at least 80%, at least 90%, at least 95%, at least 96%, at least 97%, at least 98%, or at least 99% identity with SEQ ID NO: 1, or any fragment thereof, and a pharmaceutically acceptable carrier.

In a further aspect of the invention are provided compositions comprising a polypeptide according to the invention, said composition being adapted for the detection of streptococci bacteria in or on a biological material, inanimate material or surface.

In one embodiment is provided a composition comprising a polypeptide comprising or consisting of the amino acid sequence SEQ ID NO: 1, or any variant thereof having at least 80%, at least 90%, at least 95%, at least 96%, at least 97%, at least 98%, or at least 99% identity with SEQ ID NO: 1, or any fragment thereof, wherein said variant is able to bind to the cell wall of at least one Streptococcus agalactiae bacteria (GBS).

In a particular embodiment is provided a composition for the detection of at least one Streptococcus agalactiae bacteria (GBS) comprising a polypeptide comprising the amino acid sequence SEQ ID NO: 10, or any variant thereof having at least 80%, at least 90%, at least 95%, at least 96%, at least 97%, at least 98%, or at least 99% identity with SEQ ID NO: 10, wherein said variant is able to bind to the cell wall of at least one Streptococcus agalactiae bacteria (GBS).

In a particular embodiment is provided a composition for the detection of at least one Streptococcus agalactiae bacteria (GBS) comprising the polypeptide as described above covalently linked to a reporter protein or to a radioactive, fluorescent, colorimetric, or chemiluminescent molecule.

Compositions of the invention can contain one or more lysin polypeptides. In this embodiment, lysin polypeptides can either be present as independent polypeptides or as fusion proteins comprising said lysin polypeptides or fragments thereof.

Pharmaceutical compositions of this invention may further comprise one or more pharmaceutically acceptable additional ingredient(s) such as alum, stabilizers, antimicrobial agents, buffers, coloring agents, flavoring agents, adjuvants, and the like.

The polypeptides of the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, aerosols, emulsions, elixirs, or capsules filled with the same, all for oral use, or in the form of sterile injectable solutions for parenteral (including subcutaneous) use. Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed. Compositions of this invention may also be liquid formulations including, but not limited to, aqueous or oily suspensions, solutions, emulsions, syrups, and elixirs. Liquid forms suitable for oral administration may include a suitable aqueous or non-aqueous vehicle with buffers, suspending and dispensing agents, colorants, flavors and the like. The compositions may also be formulated as a dry product for reconstitution with water or other suitable vehicle before use. Such liquid preparations may contain additives including, but not limited to, suspending agents, emulsifying agents, non-aqueous vehicles and preservatives. Suspending agents include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid. Further materials as well as processing techniques and the like are set out in Part 5 of Part 5 of Remington's “The Science and Practice of Pharmacy”, 22^{nd} Edition, 2012, University of the Sciences in Philadelphia, Lippincott Williams & Wilkins, which is incorporated herein by reference.

Solid compositions of this invention may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maizestarch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate. Tablets may be coated according to methods well known in the art.

Injectable compositions are typically based upon injectable sterile saline or phosphate-buffered saline or other injectable carriers known in the art.

Compositions of this invention may also be formulated as suppositories, which may contain suppository bases including, but not limited to, cocoa butter or glycerides. Compositions of this invention may also be formulated transdermal formulations comprising aqueous or non-aqueous vehicles including, but not limited to, creams, ointments, lotions, pastes, medicated plaster, patch, or membrane.

Compositions of this invention may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

Compositions of this invention may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

The compounds of this invention can also be administered in sustained release forms or from sustained release drug delivery systems. A description of representative sustained release materials can also be found in the incorporated materials in Remington's Remington's “The Science and Practice of Pharmacy”.

Compositions useful for decontaminating a biological material or inanimate material or surface may be formulated as solutions, aerosols or sprays, in particular aerosols.

In another aspect, the invention provides a kit comprising a polypeptide as described herewith and instructions of use, in particular for decontaminating a biological material, inanimate material or surface, or detecting the presence of a streptococci bacteria in or on said materials or surface.

In a particular aspect, is provided a kit for detecting the presence of a Gram-positive bacteria, in particular at least one streptococci bacteria selected from the group consisting of Streptococcus agalactiae (GBS), Streptococcus dysgalactiae (GCS), Streptococcus pyogenes (GAS), Streptococcus uberis, Streptococcus suis and Streptococcus gordonii, more particularly Streptococcus agalactiae (GBS), comprising a polypeptide according to the invention covalently linked to a reporter protein or to a radioactive, fluorescent, colorimetric, or chemiluminescent molecule, and instructions of use.

In a more particular aspect, the kit for detection according to the invention comprises a polypeptide comprising or consisting of SEQ ID NO:10, or any variant thereof having at least 80%, at least 90%, at least 95%, at least 96%, at least 97%, at least 98%, or at least 99% identity with SEQ ID NO: 10 capable of binding to the cell wall of Streptococcus agalactiae bacteria, wherein said polypeptide is covalently linked to a reporter protein or a radioactive, fluorescent, colorimetric, or chemiluminescent molecule.

One skilled in the art will understand that any reporter protein can be used in the kit for detection according to the invention including fluorescent proteins like Green Fluorescent Protein (GFP) and Red Fluorescent Protein (RFP), luminescent proteins like luciferase, as well as other reporter proteins like β-galactosidase, for instance. In another aspect, is provided a kit for decontaminating biological material, inanimate material or surface, comprising a polypeptide according to the invention, and instructions of use.

In a more particular aspect, the kit for decontamination according to the invention comprises a polypeptide comprising or consisting of SEQ ID NO:9, or any variant thereof having at least 80%, at least 90%, at least 95%, at least 96%, at least 97%, at least 98%, or at least 99% identity with SEQ ID NO: 9 and having an anti-bacterial activity against at least one Streptococcus agalactiae bacteria (GBS) at a pH comprised between about 7 and 8.5 at 37° C.

The kit according to the invention can further comprise reagents.

Mode of Administration

Compositions of this invention may be administered in any manner including intravenous injection, intra-arterial, intraperitoneal injection, subcutaneous injection, intramuscular, intra-thecal, oral route including sublingually or via buccal administration, topically, cutaneous application, direct tissue perfusion during surgery or combinations thereof.

The compositions of this invention may also be administered in the form of an implant, which allows slow release of the compositions as well as a slow controlled i.v. infusion. The dosage administered, as single or multiple doses, to an individual will vary depending upon a variety of factors, including pharmacokinetic properties, patient conditions and characteristics (sex, age, body weight, health, size), extent of symptoms, concurrent treatments, frequency of treatment and the effect desired.

In a particular embodiment, compounds of the invention are administered at a dose to humans of between about 1 mg to 150 mg per kg of body weight, for instance between about 10 mg and 50 mg per kg of body weight, particularly of about 45 mg per kg of body weight.

According to one aspect, the compositions of the invention may be administered in a preventive manner to patients before undergoing surgery, artificial ventilations or intubation.

Combination

According to the invention, a polypeptide according to the invention can be administered alone or in combination with a co-agent useful in the prevention and/or treatment of Gram-positive bacteria related infections or disorders, including those caused by bacteria other than Streptococcus agalactiae (GBS), Streptococcus dysgalactiae (GCS), Streptococcus pyogenes (GAS), Streptococcus uberis, Streptococcus suis and Streptococcus gordonii.

A polypeptide according to the invention can also be administered in combination with a co-agent useful in the treatment of immune-depressed patients, e.g. for example a co-agent selected from anti-HIV drug, anti-cancerous drug and the like.

The invention encompasses the administration of a polypeptide according to the invention wherein the polypeptide is administered to an individual prior to, simultaneously or sequentially with other therapeutic regimens or co-agents useful in the prevention and/or treatment of Gram-positive bacteria related infections or disorders or in the treatment of immunodepressed patients (e.g. multiple drug regimens), in a therapeutically effective amount. The polypeptide according to the invention that is administered simultaneously with said co-agents can be administered in the same or different compositions and in the same or different routes of administration. Examples of co-agents useful in the prevention and/or treatment of Gram-positive bacteria related infections or disorders include antibiotics and bacterial autolysins and other phages lysins, which possibly exhibit a different host-selectivity than the polypeptide according to the invention.

When used as a decontaminant of a biological material, inanimate material or surface, compounds of the invention can be applied to said material or surface to be decontaminated at doses of between 0.1 to 1 000 mg/l.

Patients

The invention is useful in subjects suffering from bacterial infections and disorders, in particular bacterial infections and disorders caused by Gram-positive bacteria, more particularly those caused by at least one streptococci bacteria selected from the group consisting of Streptococcus agalactiae (GBS), Streptococcus dysgalactiae (GCS), Streptococcus pyogenes (GAS), Streptococcus uberis, Streptococcus suis and Streptococcus gordonii, more particularly those caused by Streptococcus agalactiae (GBS).

In particular, the invention can be useful in subjects suffering from bacterial infections and disorders selected from the group consisting of bacteremia, meningitis, pneumonia, streptococcal toxic shock syndrome, necrotizing fasciitis, septicemia, endocarditis, deafness, mastitis.

According to one embodiment, patients according to the invention are suffering from bacteremia, in particular bacteremia caused by Streptococcus agalactiae (GBS). According to one embodiment, patients according to the invention are humans, in particular neonates (less than 4 weeks old), infants, in particular infants of less than 6 months, children, and adults including pregnant women, elderly people, adults with medical underlying conditions like diabetes, cancer, HIV infection and chronic infections.

Uses and Methods According to the Invention

In an aspect of the invention is provided a polypeptide as described herein for use as a medicament.

In another aspect of the invention, a polypeptide, compositions thereof and methods of the invention as described herein are useful for preventing and/or treating bacterial infections and disorders, in particular those caused by at least one streptococci bacteria selected from the group consisting of Streptococcus agalactiae (GBS), Streptococcus dysgalactiae (GCS), Streptococcus pyogenes (GAS), Streptococcus uberis, Streptococcus suis and Streptococcus gordonii, more particularly those caused by Streptococcus agalactiae (GBS).

In one particular embodiment is provided a polypeptide or a composition thereof as described herein for preventing and/or treating bacterial infections and disorders selected from the group consisting of bacteremia, meningitis, pneumonia, streptococcal toxic shock syndrome, necrotizing fasciitis, septicemia, endocarditis, deafness, mastitis. In a further aspect of the invention is provided a polypeptide or a composition thereof as described herein for preventing and/or treating bacteremia, in particular bacteremia caused by at least one streptococci bacteria selected from the group consisting of Streptococcus agalactiae (GBS), Streptococcus dysgalactiae (GCS), Streptococcus pyogenes (GAS), Streptococcus suis and Streptococcus gordonii, more particularly bacteremia caused by Streptococcus agalactiae (GBS).

In an alternative aspect is provided a use of a polypeptide or a composition thereof as described herein, in the manufacture of a medicament for preventing and/or treating bacterial infections and disorders, in particular those caused by at least one streptococci bacteria selected from the group consisting of Streptococcus agalactiae (GBS), Streptococcus dysgalactiae (GCS), Streptococcus pyogenes (GAS), Streptococcus uberis, Streptococcus suis and Streptococcus gordonii.

In one embodiment is provided the use of a polypeptide or a composition thereof as described herewith, in the manufacture of a medicament for preventing and/or treating bacteremia.

In an alternative aspect, is provided a method for preventing and/or treating bacterial infections and disorders, in particular those caused by at least one streptococci bacteria selected from the group consisting of Streptococcus agalactiae (GBS), Streptococcus dysgalactiae (GCS), Streptococcus pyogenes (GAS), Streptococcus uberis, Streptococcus suis and Streptococcus gordonii, more particularly those caused by Streptococcus agalactiae (GBS), comprising administering, in a subject in need thereof, a therapeutically effective amount of a polypeptide as described herein.

In one embodiment is provided a method for preventing and/or treating bacterial infections and disorders as described herewith, wherein said bacterial infections and disorders are selected from the group consisting of bacteremia, meningitis, pneumonia, streptococcal toxic shock syndrome, necrotizing fasciitis, septicemia, endocarditis, deafness, mastitis.

According to a particular embodiment, is provided a method for preventing and/or treating bacteremia.

According to a particular embodiment, bacteremia according to the invention are bacteremia caused by at least one streptococci bacteria selected from the group consisting of Streptococcus agalactiae (GBS), Streptococcus dysgalactiae (GCS), Streptococcus pyogenes (GAS), Streptococcus suis and Streptococcus gordonii, more particularly bacteremia caused by Streptococcus agalactiae (GBS).

In a still further aspect is provided a method for decontaminating a biological material including blood, inanimate material or surface, including medium such as water, air, or food, demonstrated or suspected to be infected by at least one streptococci bacteria selected from the group consisting of Streptococcus agalactiae (GBS), Streptococcus dysgalactiae (GCS), Streptococcus pyogenes (GAS), Streptococcus uberis, Streptococcus suis and Streptococcus gordonii, comprising contacting said material, surface or medium, with an effective amount of an antibacterial composition comprising a polypeptide as described herein, in particular a polypeptide comprising or consisting of SEQ ID NO: 9, or any variant thereof having at least 80%, at least 90%, at least 95%, at least 96%, at least 97%, at least 98%, or at least 99% identity with SEQ ID NO: 9, wherein said variant has an antibacterial activity (e.g. a killing activity) against at least one Streptococcus agalactiae bacteria (GBS) at a pH comprised between about 7 and 8.5, at 37° C.

In a further aspect, is provided a method for detecting the presence of at least one streptococci bacteria selected from the group consisting of Streptococcus agalactiae (GBS), Streptococcus dysgalactiae (GCS), Streptococcus pyogenes (GAS), Streptococcus uberis, Streptococcus suis and Streptococcus gordonii, in or on a biological material, inanimate material or surface, comprising:
a) contacting said biological material, inanimate material or surface, with an effective amount of a composition comprising a polypeptide as described herein, in particular a polypeptide comprising or consisting of SEQ ID NO:10, or any variant thereof having at least 80%, at least 90%, at least 95%, at least 96%, at least 97%, at least 98%, or at least 99% identity with SEQ ID NO: 10 and capable of binding to the cell wall of Streptococcus agalactiae bacteria, wherein said polypeptide is covalently linked to a reporter protein or a radioactive, fluorescent, colorimetric, or chemiluminescent molecule, and
b) detecting a signal emitted by the reporter protein or radioactive, fluorescent, colorimetric, or chemiluminescent molecule, in or on said material or surface; wherein the detection of a signal in step b) indicates the presence of said streptococci bacteria.

One skilled in the art will understand that any reporter protein can be used in the method of detection according to the invention including fluorescent proteins like Green Fluorescent Protein (GFP) and Red Fluorescent Protein (RFP), luminescent proteins like luciferase, as well as other reporter proteins like β-galactosidase, for instance. The measurement of fluorescence can be carried out by any standard method including, for instance, fluorescent spectroscopy, fluorescence microscopy, and FACS. The measurement of luminescence can be carried out by any standard method including luminescent spectroscopy, luminescence microscopy.

References cited herein are hereby incorporated by reference in their entirety. The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

The invention having been described, the following examples are presented by way of illustration, and not limitation.

### EXAMPLES

The following abbreviations refer respectively to the definitions below:

aa (amino acid); by (base pair), cm (centimeter), h (hour), i.p. (intraperitoneally), μl (microliter), μM (micromolar), mM (millimolar), mg (milligram), min (minute), nm (nanometer), rpm (rotation per minute), CFU (colony-forming unit), OD₆₀₀ₙₘ (Optical density measured at 600 nm), MurNac-LAA (amidase catalytic domain), CHAP (cysteine histidine-dependent aminohydrolase/peptidase catalytic domain), GH25 (glycosyl hydrolase family 25 catalytic domain), LA (lysogeny broth agar), LB (lysogeny broth), LysM (LysM cell wall binding domain) and SH3 (SH3 cell wall binding domain), plySK1249 (lysin polypeptide isolated from Streptococcus dysgalactiae subsp. equisimilis (SDSE) strain SK1249), PBS (Phosphate Buffer Sulfate).

Materials and Methods

Bacterial Strains and Reagents

If not specified, chemicals used herewith were purchased from Sigma-Aldrich (Saint Louis, Mo., USA). Restriction enzymes were obtained from Promega (Madison, Wis., USA) and primers were synthesized by Microsynth AG (Balgach, Switzerland). For bacterial cultures, Difco™ dehydrated media (Becton Dickinson, Sparks, Md., USA) were used and reconstituted with demineralized water at 80.

The strains used herewith were as follows: E. coli BL21(DE3)pLysS (F-ompT hsdSB (rB-mB) dcm+ gal (DE3) pLysS (CamR)) obtained from Stratagene, SDSE SK1249 isolated from a human hemoculture (Vandamme et al, 1996, Int J Syst Bacteriol 46:774-781, 1996, CCUG 36637), GBS FSL-S3-026 from bovine source (Richards et al., 2011, 1 Infection, Genetics and Evolution, 11(6): 1263-1275), GBS 17-2167 isolated from a human endocarditis sample, GBS 532 from a human sample, GBS GF isolated from a human hemoculture, S. pyogenes ATCC 19615 isolated from a human sore throat sample, S. gordonii DL1 from human source (Kolenbrander et al, 1990, Appl Environ Microbiol. 56: 3890-3894), S. mutans ATCC 25175 isolated from a human carious dentine sample, S. suis #19 from porcine source, S. uberis ATCC 700407 from bovine source, S. mutans ATCC 25175 isolated from a human carious dentine sample, E. faecalis ATCC 29212 isolated from human urine sample, E. faecium D344 from human (Williamson et al, 1985, J Gen Microbiol. 131:1933-1940), S. aureus M32 isolated from a the milk of a cow suffering from subclinical mastitis (Sakwinska et al, 2011, Appl Environ Microbiol. 77(17):5908-15), S. aureus Laus102 isolated from a human healthy patient (Sakwinska et al, 2011, supra), E. faecalis ATCC 29212 isolated from a human urine sample.

All strains used herewith were grown aerobically at 37° C. and 250 r.p.m agitation with the exception of S. pneumoniae, which was grown without agitation. Streptococcal and pseudomonas strains were cultured in brain heart infusion (BHI) and plated on Mueller-Hinton agar containing 5% sheep blood (bioMérieux S A, Marcy l'Etoile, France) or BHI agar, respectively. S. aureus was grown in tryptic soy broth (TSB) and plated as streptococci. Escherichia coli strains were grown in lysogeny broth (LB) and plated on lysogeny broth agar (LA). Frozen stocks were made from cultures in exponential phase of growth supplemented with 20% glycerol (vol/vol).

Cloning of plySK1249

Chromosomal DNA was prepared from SDSE strain SK1249 using the DNeasy Blood & Tissue Kit (Qiagen, Valencia, Calif., USA) according to the manufacturer's instructions. plySK1249 was PCR amplified with specific primers, digested with corresponding restrictions endonucleases and ligated into expression vectors (Merck KGaA, Darmstadt, Germany). pET-15b and pET-28a expression vectors were chosen in order to obtain constructs with His-tag at N-terminus and C-terminus of plySK1249, respectively.

SEQ ID NO: 3: plySK15bNdeI forward primer
GGAATTCCATATGGGAAAACATCTAGTCATTTGTGGTCATGGGCAAGGG
CG
SEQ ID NO: 4: plySK15bBamHI reverse primer
CGCGGATCCTTAATGAAATTCTAAACCAACCAACAACTTTTCCAAGTTT
AACTGTTCCAG
SEQ ID NO: 5: plySK28aNcoI forward primer
GCATGCCATGGGAAAACATCTAGTGATTTGTGGACATGGGCAAGGACG
SEQ ID NO: 6: plySK28aXhoI reverse primer
GCCGCTCGAGTGAAATTCTAAACCAACCTACAACTTTTCCAAGTTTAAC
TGTTCCAG

Constructs were transformed in One Shot® BL21(DE3)pLysS Chemically Competent E. coli cells (Life Technologies Europe B.V., Zug, Switzerland) and validated by DNA sequencing using the pET vectors universal T7 forward and reverse primers.

SEQ ID NO: 7: Universal T7 forward primer
TAATACGACTCACTATAGGG
SEQ ID NO: 8: Universal T7 reverse primer
GCTAGTTATTGCTCAGCGG

plySK1249 Expression and Activity Screening of the Gene Product

In order to check for the expression of the lysin gene and screen for the antibacterial activity of PlySK1249, a protocol was adapted from Schmitz et al., 2010, Appl Environ Microbiol., 76(21):7181-7. Briefly, BL21(DE3)pLysS/Ply SK1249^{15b} and BL21(DE3)plysS/PlySK1249^{28a} transformants were replica plated on LA plates supplemented with kanamycin (30 μg/ml), chloramphenicol (25 μg/ml), and 0.4 mM isopropyl-1-thio-β-D-galactopyranoside (IPTG). Following overnight growth at 37° C., the colonies were exposed to chloroform vapors for 20 min to permeabilize the cells. Colonies were further overlaid with 15 ml of molten soft agar containing autoclaved SDSE strain SK1249 cells. Plates were incubated at 37° C. and observed for clearing zones surrounding colonies for up to 24 h. To prepare molten soft agar, an overnight culture of SDSE strain SK1249 was centrifuged, washed with 1 volume of NaCl 0.9% and resuspended in 0.25 vol of PBS pH 7.4. The cell suspension was further supplemented with granulated agar (7.5 g/L), autoclaved for 15 min at 120° C., and stored at 4° C. Before use, the agar was melted in a microwave and equilibrated in a water bath set to 55° C.

Purification of PlySK1249

A starter culture from an E. coli BL21(DE3)plysS/PlySK1249^{28a} colony surrounded by a large lysis zone in the screening test was grown overnight in LB supplemented with kanamycin (30 μg/ml) and chloramphenicol (50 μg/ml). On the following morning, the culture was diluted with 20 volumes of pre-heated fresh LB and further grown at 37° C. and 220 rpm. At OD₆₀₀ₙₘ of 0.7, the culture was induced for 20 h at 18° C. by the addition of 0.4 mM Isopropyl β-D-1-thiogalactopyranoside (IPTG). Cells were centrifuged, washed with 30 ml of NaCl 0.9%, and resuspended in 30 ml of binding buffer (20 mM imidazole, 20 mM phosphate buffer, 0.5 M NaCl, pH 7.4). Aliquots of 15 ml were prepared in 50 ml Falcon® tubes and frozen overnight at −80° C. The aliquots were thawed and sonicated on ice (Sonopuls, Bandelin electronics, Berlin, Germany). Cell debris was removed from pooled supernatants by centrifugation (15,000 rpm, 30 min, 4° C.). Supernatant were further treated with 1 μg/ml of RNAse A and DNAse I (Roche AG, Basel, Switzerland) for 45 min at 4° C., and filtered through 0.45 Acrodisc filters (Pall, Ann Arbor, USA). The filtrate was applied to a 5 ml HisTrap HP column (GE Healthcare, Glattburgg, Switzerland) previously equilibrated with binding buffer and coupled to an ÄKTA Prime apparatus (GE Healthcare). Following a washing step with 50 ml of 50 mM imidazole, 20 mM phosphate buffer, 0.5 M NaCl, pH 7.4, His-tagged PlySK1249 was eluted with 500 mM imidazole, 20 mM phosphate buffer, 0.5 M NaCl, pH 7.4. Imidazole was removed by extensive dialysis against lysin buffer (500 mM L-arginine, 50 mM phosphate buffer, pH 7.4) using a membrane tubing (MWCO 12-14,000 Da, Spectra/Por®, Rancho Dominguez, Calif., USA). PlySK1249 was migrated on NuPage 4-12% Bis-Tris gels (Invitrogen, Carlsbad, Calif., USA) and protein bands were compared to the Novex® Sharp Standard (Invitrogen) for molecular weight determination.

In Vitro Quantification of the Antibacterial Activity of PlySK1249, Effect of pH and Bacterial Growth Phases

PlySK1249 activity was measured by following the decrease in turbidity of a solution of SDSE cells resuspended in lysis buffer (40 mM phosphate buffer, 200 mM NaCl, pH 7.4). Briefly, bacterial cells were grown until an Optical Density at 600 nm (OD₆₀₀ₙₘ) of about 0.4 is reached, and harvested by centrifugation before being washed with NaCl 0.9% and resuspended in lysis buffer to an OD₆₀₀ₙₘ of 1. Activity was measured by mixing 150 μl of the bacterial cell suspension with 150 μl of serial two-fold dilutions of PlySK1249 in 96-wells microtiter plates. Serial dilutions of the enzyme were done in lysis buffer. The decrease in OD₆₀₀ₙₘ was immediately monitored with an EL808 absorbance microplate reader run with Gen5™ software (BioTek, Winooski, Vt., USA) set to 37° C. The OD₆₀₀ₙₘ was read every min over a period of 1 h. The well in which a decrease of the optical density by half in 15 min was observed, was defined as containing one unit (1 U) of purified enzyme (Loeffler et al, 2003, Infect Immun. 2003; 71 (11): 6199-204).

In order to test the effect of the pH on the PlySK1249 activity, SDSE cells suspensions and PlySK1249 solutions were both prepared in buffers with pH values ranging from 4.0 to 9.0. In order to test the effect of the growth phases on the sensitivity of SDSE to PlySK1249, cells were harvested at OD₆₀₀ₙₘ of 0.13, 0.48, 0.85, and 1.02, before being further processed as described above. All reactions were performed in triplicate. The percentage of decrease in turbidity was calculated by the following formula: 100−((ODf*100)/ODi) with ODf is the final OD₆₀₀ₙₘ and ODi is the initial OD₆₀₀ₙₘ.

In Vitro Time-Kill Assays

To determine bacterial viability, time-kill assays were performed in triplicate by challenging either a solution of SDSE strain SK1249 at 10⁹ CFU/ml or a solution of GBS clinical strain 17-2167 at 5.10⁸ CFU/ml resuspended in lysis buffer with 3.3 U/ml of PlySK1249 at 37° C. At different incubation times over a total period of 1 h, 100 μl aliquots were taken, serially diluted in 10 ml ice cold NaCl 0.9%, and plated for numeration of viable bacterial cells.

PlySK1249 Therapeutic Trials in Mice

The mouse model of GBS bacteremia was used to test the activity of the lysin polypeptide of the invention. A total of 35 six-weeks-old CD1® Swiss female mice (Charles River Laboratories, L'Arbresle, France) with an average weight of 22±1 g were used herewith. In order to validate the bacteremic state of mice at the time of the initial treatment injection, i.e. 1 h after the bacterial challenge, left-side kidney and spleen were removed aseptically from 3 mice 45 min. after i.p injection of 10⁶ CFU of GBS clinical strain 17-2167. Organs were homogenized in 1 ml of saline, briefly centrifuged, and supernatants were serially diluted before being plated on blood agar plates. Plates were incubated for 48 h at 37° C. to determine the number of viable organisms in the tissues. For therapeutic experiments, animal sample sizes were estimated with the formula for dichotomous variables (Dell et al, 2002, ILAR J. 43(4):207-13). In a first series of experiments conducted in order to evaluate the effect of a single bolus injection of PlySK1249, two groups of mice were injected i.p. with 10⁶ CFU of the GBS clinical strain 17-2167. While the first group (n=8) received 22.5 mg/kg of PlySK1249 in 100 μl i.p. 1 h after the bacterial challenge, the second group received 100 μl of lysin buffer i.p (n=7). In a second series of experiment conducted in order to test a different dosage of the treatment, two groups of mice were injected i.p. with 10⁶ CFU of the GBS clinical strain 17-2167. While the first group (n=10) received 45 mg/kg of PlySK1249 in 200 μl i.p. at 2, 20, and 26 h after the bacterial challenge, the second group (n=10) received 200 μl of lysin buffer i.p. at the same times. The percentage of Survival was calculated as (Ns*100)/N with Ns is the number of surviving mice and N is the initial number of mice. Mice were monitored for survival over a period of five days, and results were plotted in Kaplan-Meier survival curves, analyzed, and compared with Log-Rank (Mantel-Cox) and Gehan-Breslow-Wilcoxon tests using GraphPad Prism version 5.00 for Windows (GraphPad Software, San Diego, Calif., USA, www.graphpad.com).

### Example 1

### Cloning, Expression, Purification and Structure of the plySK1249 Lysin Cloning and Expression Screening

plySK1249 was successfully amplified by PCR from purified genomic DNA of SDSE SK1249 and cloned into both pET-15b and pET-28a expression vectors leading to the constructs PlySK1249^{15b} and PlySK1249^{28a}, respectively. Following IPTG-induction, chloroform permeabilization, and overlay with soft agar containing autoclaved SDSE SK1249 cells, lysis zones developed around BL21/PlySK1249^{28a} colonies. In contrast, no lysis zones could be observed around BL21/PlySK1249^{15b} colonies. A single BL21/PlySK1249^{28a} colony, surrounded by a large halo, was selected and further grown in order to purify PlySK1249.

Purification

About 90% purity was achieved in a single step purification process on a 5 ml HisTrap HP column. On a 4-12% Novex® Bis-Tris gel, purified PlySK1249 migrated at the expected molecular weight of 53 kDa (not shown). PlySK1249 was stored at −20° C. in lysin buffer (500 mM L-arginine, 50 mM phosphate buffer, pH 7.4) until further use.

Structure

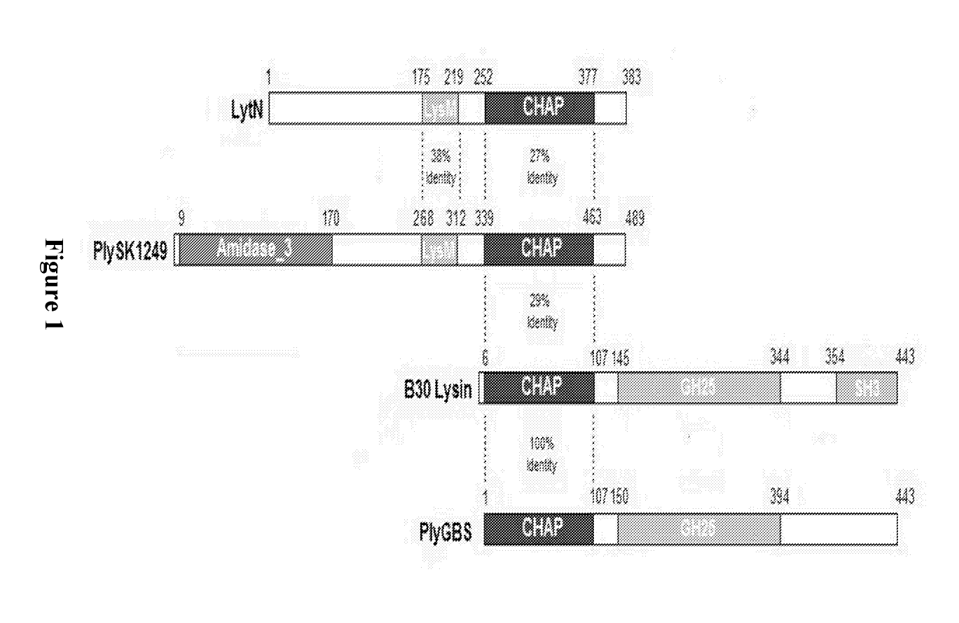

Conserved domains analysis carried out on the amino acid sequence SEQ ID NO: 1 of PlySK1249 indicated that PlySK1249 harbours three different domains (FIG. 1), i.e. i) a N-terminal catalytic domain with a predicted amidase activity that is also present in several uncharacterized proteins found in different strains of S. dysgalactiae, GBS, and S. suis; ii) a central LysM domain with a predicted cell wall-binding activity that shares 38% identity with a similar domain found in the S. aureus LytN autolysin (Frankel et al, 2011, 1 Biol. Chem., 286(37):32593-605), and a NLPC_P60 C-terminal domain that shares 27% and 29% identity with the cysteine histidine-dependent aminohydrolase/peptidase (CHAP) domains of the LytN autolysin and the GBS B30 lysin (Pritchard et al. 2004, supra; Baker et al. 2006, supra), respectively. The conserved cysteine and histidine residues of the CHAP domain were found at positions 353 and 414 of PlySK1249 sequence, respectively

### Example 2

### In Vitro Measurement of PlySK1249 Antibacterial Activity and Evidence of its Killing Activity

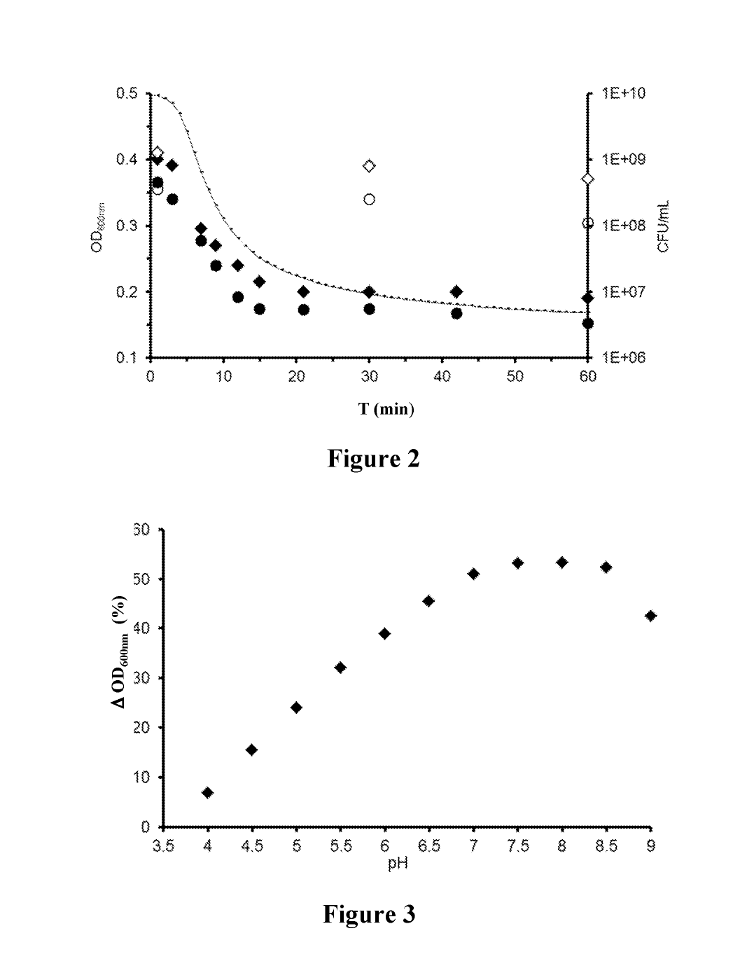

The antibacterial activity of a purified lysin is commonly quantified by following its capacity to decrease the turbidity of a suspension of bacterial cells over time. In this type of turbidity assay, 1 U of enzyme is defined as the amount of enzyme that could decrease by half in 15 min and 37° C. the OD₆₀₀ₙₘ of a 300 μl suspension of bacterial cells that were harvested in the mid-logarithmic growth phase. Using this commonly admitted definition, purified PlySK1249 showed a specific activity of 1 U/μg or 50 U/nmol against SDSE strain SK1249 (FIG. 2). In order to determine if the observed loss of turbidity resulted from cell burst and subsequent death, bacterial cell viability was further tested when exposed to PlySK1249 in time-kill experiments. To this end, a suspension of SDSE SK1249 harvested in mid-log phase was challenged with 3.3 U/ml of lysin and plated for numeration at different incubation times (FIG. 2). The data showed an about 2 log CFU/ml decrease in 15 min, which was in agreement with the observed decrease in turbidity. Similarly, about 2 log CFU/ml decrease in 15 min was achieved with the same amount of PlySK1249 for the GBS strain 2167 which has been used in the mouse model of GBS-induced bacteremia (FIG. 2).

In summary, the decrease in turbidity (OD₆₀₀ₙₘ) of SDSE and GBS cell suspensions was shown to be correlated with a decrease in cells viability. Indeed, 1 U of PlySK1249 not only decreased OD₆₀₀ₙₘ by half but also CFU/ml of a solution of SDSE SK1249 or GBS stain 17-2167 harvested in mid-exponential phase by about 2 log₁₀ in 15 min. In comparison, 40 U of PlyGBS were required to decrease the cell viability of a suspension of GBS strain NCTC 11237 cells by about 2 log₁₀ in 40 min (Cheng et al, 2005, supra).

### Example 3

### Effect of pH and Bacterial Growth Phase on PlySK1249 Antimicrobial Activity

Enzymes are affected by pH changes and extreme values generally result in loss of activity. A pH profile was determined for PlySK1249, and the optimum pH was found between pH 7 and 8.5 (FIG. 3) with about 50% of OD₆₀₀ₙₘ decrease achieved at these pH values. Activity was significantly reduced at acidic and basic pH with, as instance, only 5% OD₆₀₀ₙₘ decrease at pH 4.

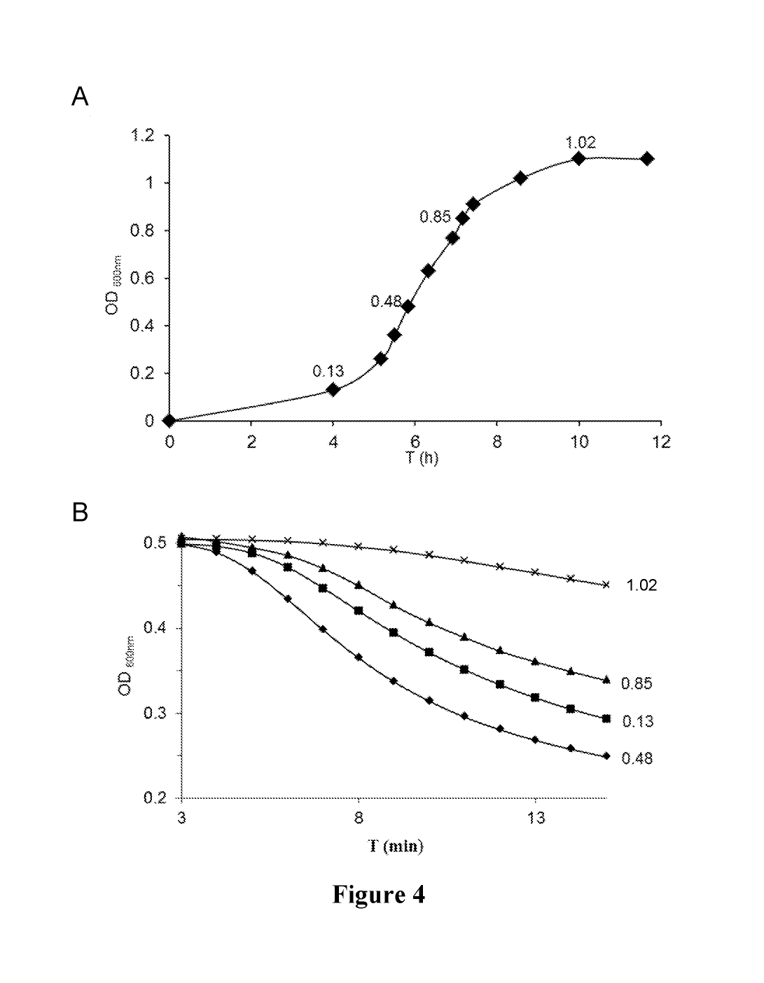

It is well-known that the activity of phage lysins is dependent of the growth phase in which the bacterial targets are when challenged by the enzyme. In order to test the sensitivity of bacterial cells to PlySK1249, aliquots of SDSE strain SK1249 were harvested at different stages during the growth (FIG. 4A) and further challenged by purified PlySK1249 in turbidity assays (FIG. 4B). Bacterial cells harvested at early and mid-exponential phases were much more sensitive to PlySK1249 than cells harvested in the late exponential or stationary phases. As instance, while the turbidity of a solution of cells harvested in the exponential growth phase (i.e. at OD₆₀₀ₙₘ of 0.48) decreased by half in 15 min., this decrease was only of <5% with cells harvested in the early stationary growth phase (i.e. at OD₆₀₀ₙₘ of 1.02 (FIG. 4B).

In summary, the optimum pH of PlySK1249 was found to be between pH 7 and 8.5. In comparison, the B30 lysin from Group B streptococcal bacteriophage B30 has been found to have a low rate of lysis on Group B streptocci with an optimum pH ranging between 5.5 and 6 (Pritchard et al. 2004, supra). On the other hand, PlyGBS, isolated from the GBS phage NCTC 11261 and having 99% homology with B30 lysin, is active against group A, B, C, G and L streptococci and has an optimum pH about 5 (Cheng et al., 2005, Antimicrobial Agents and Chemotherapy, 49(1): 111-117).

### Example 4

### Host Range of PlySK1249

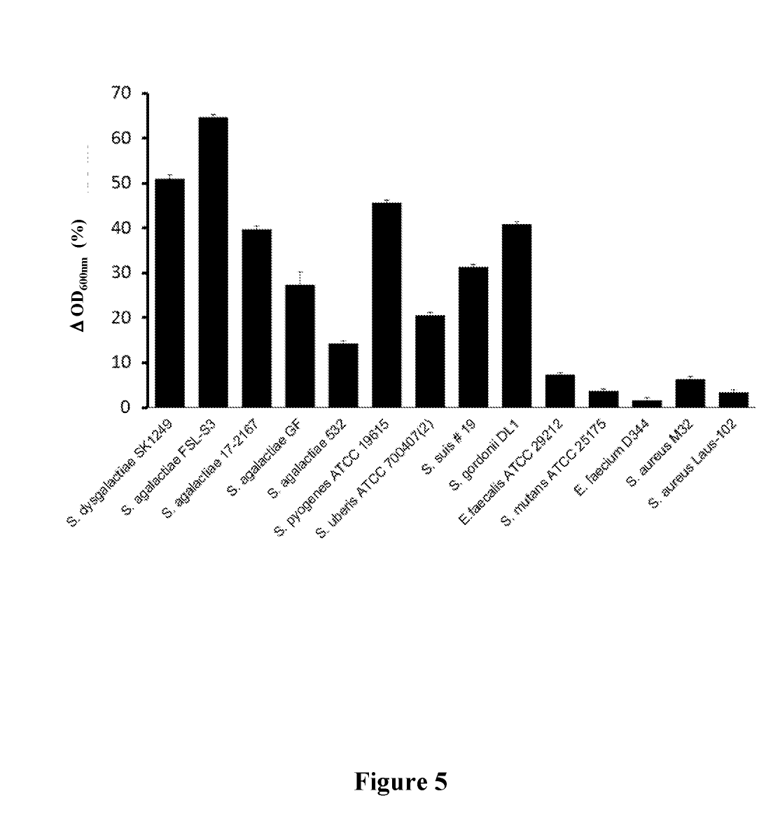

In order to determine the activity spectrum of PlySK1249, the sensitivity of several different bacterial species was tested in turbidity assays. All β-haemolytic species tested (S. dysgalactiae, GBS, and Streptococcus pyogenes) were sensitive to PlySK1249, with strain-to-strain variation for GBS (FIG. 5). Indeed, while about 65% turbidity decrease was observed for the GBS strain FSL-03, only an about 15% decrease was achieved in the same conditions with strain 532. Interestingly, Streptococcus uberis and Streptococcus suis were also sensitive with about 20% and about 30% decrease in turbidity, respectively. PlySK1249 had a good activity against Streptococcus gordonii strain DL1 (about 45% turbidity decrease), but negligible against Streptococcus mutans, S. aureus, and Enterococcus faecalis (<10% turbidity decrease in all cases) (FIG. 5). In summary, lytic activity of PlySK1249 was demonstrated in vitro against S. dysgalactiae and GBS, as well as against S. pyogenes, which is the common etiologic agent of pharyngitis and life threatening infections such as streptococcal toxic shock syndrome, necrotizing fascitis or septicaemia and against S. uberis, the most common streptococcal species isolated from cow mastitis, and S. suis, a major pig and emerging zoonotic pathogen. At the exception of S. gordonii, no significant activity was observed for PlySK1249 towards other commensal streptococcus of the oral and intestinal cavity. Therefore, and since E. faecalis and E. faecium are the predominant Gram-positive bacteria of the human gut flora, this observation could preclude that the use of this lysin in therapy would not induce major intestinal side effects.

### Example 5

### PlySK1249 Efficacy in a Mouse Model of GBS-Induced Bacteremia

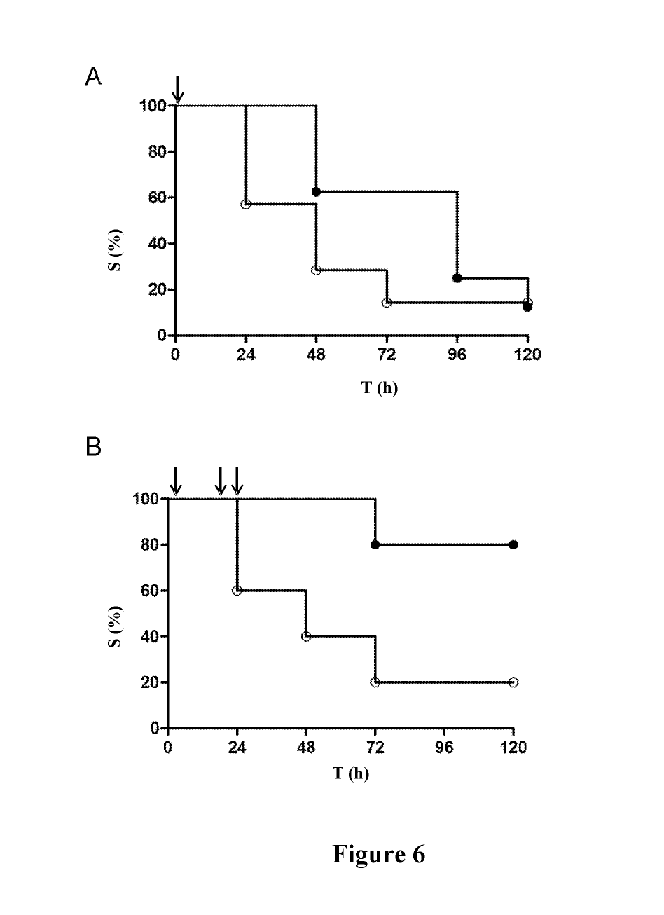

The therapeutic potential of PlySK1249 was tested in a mouse model of GBS-induced bacteremia. To establish the time for first injection of the treatment, three CD1® Swiss female mice (22±1 g) were infected i.p. with 10⁶ CFU of GBS clinical strain 17-2167 in 100 μL of NaCl 0.9%. 1 h post-infection, animals were euthanized and various organs tested for viable GBS. More than 10⁵ CFU/g bacteria were found in the spleen and kidneys demonstrating the animals were bacteremic already at this time point. Thus, first bolus of PlySK1249 was administered at least 1 h after the bacterial challenge in subsequent experiments. In a first series of experiment, CD1® Swiss female mice (22±1 grams) were infected by i.p. injection of 10⁶ GBS strain 2167 in 100 μL of NaCl 0.9%. 1 h after the bacterial challenge mice were injected i.p. either with 100 μL of lysin buffer (n=7) or a single bolus of 22.5 mg/kg PlySK1249 in 100 μL lysin buffer (n=8). PlySK1249 treatment slightly shifted the survival curve to the right suggesting delay of death, at least at 24 h for some animals (FIG. 6A). Increasing the dose of PlySK1249 to 135 mg/kg injected in three bolus within the first 24 h post-infection (i.e. 45 mg/kg at 2, 20, and 24 h) had a significant effect (p<0.01) on the mice survival (FIG. 6B). Indeed, while about 80% of mice died within 5 days post-infection in the control group receiving only lysin buffer (n=10), 80% of mice survived within the same period of time in the group treated with repeated injections of PlySK1249 (n=10). This significant result demonstrated the therapeutic efficacy of PlySK1249 in a model of GBS-induced bacteremia.

Taken together these results demonstrate that, in comparison to other bacterial lysins of the prior art, PlySK1249 exhibits particular properties, including an antibacterial activity against GBS observed at an optimal pH comprised between about 7 and 8.5, that makes a composition comprising said lysin particularly suitable for the treatment of bacteremia caused by GBS.

List of Sequences

SEQ ID NO: 1: lysin polypeptide (Streptococcus
dysgalactiae subsp. equisimilis
(SDSE) strain SK1249) (489 amino acids)
MGKHLVICGHGQGRTTYDPGAVNAKLGITEAGKVRELAKLMSKYSGQQI
DFITEQNVYDYRSITSIGKGYDSITELHFNAFNGSAKGTEVLIQSSLEA
DKEDMAILSLLSRYFQNRGIKKVDWLYNANQAASRGYTYRLVEIAFIDN
EQDMAIFETKKEDIAKGLVSAITGVEVKTIVPSTPSSTVGSSGTPSKPI
YLVGDSLRVLPHATHYQTGQKIANWVKGRTYKILQEKNVHQSNSLRAYL
LDGIKSWVLEQDVEGTTKGHSEQTYQAQKGDTYYGIARKFGLTVDALLA
VNGLKKTDILRVGQTLKVNAASRITTAIPTSVASRVVASALSKVGQKVT
VPSNPYGGQCVALVDKIVQELTDKNMSYTNAIDCLKKAKSNGFQVIYDA
WGVNPKAGDFYVIETDGLVYGHIGVCVTDSDGKSIDGVEQNIDGYSDHN
KNGINDQLEIGGGGITRRVKRQWMADGSLYDSTGTVKLGKVVGWFRIS
SEQ ID NO: 2: nucleotide sequence encoding the
above lysin polypeptide (Streptococcus
dysgalactiae subsp. equisimilis (SDSE) strain
SK1249)
ATGGGAAAACATCTAGTGATTTGTGGACATGGGCAAGGACGAACGACCT
ATGATCCAGGTGCAGTAAATGCCAAACTAGGCATCACAGAAGCAGGAAA
GGTTCGAGAATTAGCCAAGTTAATGTCTAAGTACAGTGGACAACAGAT
TGATTTTATTACCGAACAAAATGTTTATGATTATCGGAGTATTACTAGT
ATTGGTAAGGGATACGACTCAATTACTGAATTGCACTTCAATGCCTTTA
ATGGTAGTGCCAAAGGTACAGAAGTCTTGATTCAATCTTCTTTAGAAGC
AGACAAGGAAGATATGGCTATCCTCTCTCTCCTTTCACGATACTTTCAA
AATCGTGGCATTAAGAAGGTAGATTGGCTCTATAATGCCAACCAAGCAG
CGAGTCGTGGATATACCTATCGTTTGGTGGAGATTGCCTTTATCGATAA
TGAACAAGACATGGCGATTTTTGAAACCAAGAAAGAGGACATTGCGAAA
GGTCTTGTGTCCGCAATAACAGGGGTTGAGGTCAAGACAATTGTACCCT
CGACACCCAGTTCAACTGTTGGGAGTTCAGGAACTCCTTCAAAACCAAT
CTATCTTGTTGGTGATAGTCTTAGGGTGTTGCCTCATGCGACTCATTAT
CAGACTGGTCAGAAAATCGCCAATTGGGTCAAGGGGCGCACCTACAAAA
TCCTCCAAGAAAAAAATGTTCACCAGTCTAACAGTTTGAGAGCTTATCT
ACTTGATGGAATCAAGTCATGGGTGCTGGGCAGGATGTAGAAGGAACAA
CTAAAGGCCATAGTGAGCAGACCTATCAAGCACAGAAAGGCGATACGTA
TTATGGTATCGCTCGGAAGTTTGGTTTAACAGTTGATGCCCTTCTTGCG
GTAAATGGCTTGAAGAAGACGGATATTTTAAGAGTTGGACAAACTCTAA
AGGTCAACGCAGCTTCAAGGATAACAACCGCTATTCCAACCAGTGTTGC
AAGCCGTGTGGTTGCGTCAGCATTATCCAAGGTCGGTCAAAAGGTAACT
GTTCCATCTAACCCTTATGGTGGACAGTGTGTTGCCTTGGTGGATAAGA
TTGTTCAAGAACTTACGGATAAGAATATGTCCTATACAAATGCCATTGA
TTGTTTGAAGAAAGCAAAATCAAATGGTTTCCAAGTAATCTATGATGCT
TGGGGTGTGAATCCTAAAGCAGGTGATTTCTATGTCATTGAGACAGATG
GTTTGGTCTATGGGCATATTGGTGTCTGTGTGACGGATTCTGATGGAAA
AAGTATTGATGGTGTGGAACAGAATATTGACGGATATTCTGACCATAAT
AAGAACGGTATCAATGACCAATTAGAAATTGGTGGAGGTGGAATTACTC
GTCGTGTGAAACGTCAATGGATGGCGGATGGCTCACTCTATGATTCTAC
TGGAACAGTTAAACTTGGAAAAGTTGTAGGTTGGTTTAGAATTTCATAA
SEQ ID NO: 3: plySK15bNdeI forward primer
(artificial sequence)
GGAATTCCATATGGGAAAACATCTAGTCATTTGTGGTCATGGGCAAGGG
CG
SEQ ID NO: 4: plySK15bBamHI reverse primer
(artificial sequence)
CGCGGATCCTTAATGAAATTCTAAACCAACCAACAACTTTTCCAAGTTT
AACTGTTCCAG
SEQ ID NO: 5: plySK28aNcoI forward primer
(artificial sequence)
GCATGCCATGGGAAAACATCTAGTGATTTGTGGACATGGGCAAGGACG
SEQ ID NO: 6: plySK28aXhoI reverse primer
(artificial sequence)
GCCGCTCGAGTGAAATTCTAAACCAACCTACAACTTTTCCAAGTTTAAC
TGTTCCAG
SEQ ID NO: 7: Universal T7 forward primer
(artificial sequence)
TAATACGACTCACTATAGGG
SEQ ID NO: 8: Universal T7 reverse primer
(artificial sequence)
GCTAGTTATTGCTCAGCGG
SEQ ID NO: 9: Amidase 3 active domain of
plysSK1249
GHGQGRTTYDPGAVNAKLGITEAGKVRELAKLMSKYSGQQIDFITEQNV
YDYRSITSIGKGYDSITELHFNAFNGSAKGTEVLIQSSLEADKEDMAIL
SLLSRYFQNRGIKKVDWLYNANQAASRGYTYRLVEIAFIDNEQDMAIFE
TKKEDIAKGLVSAIT
SEQ ID NO: 10: LysM of plysSK1249
QTYQAQKGDTYYGIARKFGLTVDALLAVNGLKKTDILRVGQTLKV

1-17. (canceled)

18. An antibacterial composition comprising a polypeptide comprising the amino acid sequence SEQ ID NO: 1, or any variant thereof having at least 80% identity with SEQ ID NO: 1, or any fragment thereof comprising SEQ ID NO: 9, or any variant thereof having at least 80% identity with SEQ ID NO: 9.

19. An antibacterial composition according to claim 18, wherein said polypeptide has a killing activity against at least one Streptococcus agalactiae bacteria (GBS) at a pH comprised between about 7 and 8.5, at 37° C.

20. The antibacterial composition according to claim 18, wherein the polypeptide has the amino acid sequence of SEQ ID NO: 1.

21. The antibacterial composition according to claim 18, wherein said polypeptide further has a killing activity against at least one streptococci bacteria selected from the group consisting of Streptococcus dysgalactiae (GCS), Streptococcus pyogenes (GAS), Streptococcus uberis, Streptococcus suis and Streptococcus gordonii.

22. The antibacterial composition according to claim 18, wherein said composition is a pharmaceutical composition further comprising a pharmaceutically acceptable carrier.

23. A method for decontaminating a biological material, an inanimate material or surface, demonstrated or suspected to be infected by at least one streptococci bacteria selected from the group consisting of Streptococcus agalactiae (GBS), Streptococcus dysgalactiae (GCS), Streptococcus pyogenes(GAS), Streptococcus uberis, Streptococcus suis and Streptococcus gordonii, comprising a step of contacting said material or surface with an effective amount of a polypeptide comprising the amino acid sequence SEQ ID NO: 1, or any variant thereof having at least 80% identity with SEQ ID NO: 1, or any fragment thereof comprising SEQ ID NO: 9, or any variant thereof having at least 80% identity with SEQ ID NO: 9, or a composition thereof, wherein said variant or fragment has a killing activity against at least one Streptococcus agalactiae bacteria (GBS) at a pH comprised between 7 and 8.5, at 37° C.

24. A method for preventing and/or treating bacterial infections and disorders comprising administering, in a subject in need thereof, a therapeutically effective amount of a polypeptide comprising the amino acid sequence SEQ ID NO: 1, or any variant thereof having at least 80% identity with SEQ ID NO: 1, or any fragment thereof comprising SEQ ID NO: 9, or any variant thereof having at least 80% identity with SEQ ID NO: 9, or a composition thereof.

25. A method according to claim 24, wherein said bacterial infections and disorders are caused by at least one streptococci bacteria selected from the group consisting of Streptococcus agalactiae (GBS), Streptococcus dysgalactiae(GCS), Streptococcus pyogenes (GAS), Streptococcus uberis, Streptococcus suis and Streptococcus gordonii.

26. A method according to claim 24, wherein said bacterial infections and disorders are selected from the group consisting of bacteremia, meningitis, pneumonia, streptococcal toxic shock syndrome, necrotizing fasciitis, septicemia, endocarditis, deafness, and mastitis.

27. A method according to claim 24, wherein the said bacterial infection and disorder is bacteremia.

28. A method according to claim 24, wherein said bacterial infections and disorders are bacteremia caused by Streptococcus agalactiae (GBS).

29. A method for detecting the presence of at least one streptococci bacteria selected from the group consisting of Streptococcus agalactiae (GBS), Streptococcus dysgalactiae (GCS), Streptococcus pyogenes (GAS), Streptococcus uberis, Streptococcus suis and Streptococcus gordonii, in or on a biological material, inanimate material or surface, comprising:
a) contacting said material or surface with an effective amount of a composition comprising a polypeptide comprising or consisting of SEQ ID NO:10, or any variant thereof having at least 80%, at least 90%, at least 95%, at least 96%, at least 97%, at least 98%, or at least 99% identity with SEQ ID NO: 10 and capable of binding to the cell wall of Streptococcus agalactiae bacteria, wherein said polypeptide is covalently linked to a reporter protein or a radioactive, fluorescent, colorimetric, or chemiluminescent molecule, and
b) detecting a signal emitted by the reporter protein or radioactive, fluorescent, colorimetric, or chemiluminescent molecule, in or on said material or surface; wherein the detection of a signal in step b) indicates the presence of said streptococci bacteria.

30. A kit for decontaminating biological material, inanimate material or surface, comprising a polypeptide comprising or consisting of SEQ ID NO:9, or any variant thereof having at least 80%, at least 90%, at least 95%, at least 96%, at least 97%, at least 98%, or at least 99% identity with SEQ ID NO: 9 and having an anti-bacterial activity against at least one Streptococcus agalactiae bacteria (GBS) at a pH comprised between about 7 and 8.5 at 37° C., and instructions of use.


SEQUENCE LISTING (10 sequences)

SEQ ID NO: 1 - protein, 489 aa, Streptococcus dysgalactiae
MGKHLVICGHGQGRTTYDPGAVNAKLGITEAGKVRELAKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFETKKEDIAKGLVSAITGVEVKTIVPSTPSSTVGSSGTPSKPIYLVGDSLRVLPHATHYQTGQKIANWVKGRTYKILQEKNVHQSNSLRAYLLDGIKSWVLEQDVEGTTKGHSEQTYQAQKGDTYYGIARKFGLTVDALLAVNGLKKTDILRVGQTLKVNAASRITTAIPTSVASRVVASALSKVGQKVTVPSNPYGGQCVALVDKIVQELTDKNMSYTNAIDCLKKAKSNGFQVIYDAWGVNPKAGDFYVIETDGLVYGHIGVCVTDSDGKSIDGVEQNIDGYSDHNKNGINDQLEIGGGGITRRVKRQWMADGSLYDSTGTVKLGKVVGWFRIS

SEQ ID NO: 2 - DNA, 1469 bp, Streptococcus dysgalactiae
atgggaaaacatctagtgatttgtggacatgggcaaggacgaacgacctatgatccaggtgcagtaaatgccaaactaggcatcacagaagcaggaaaggttcgagaattagccaagttaatgtctaagtacagtggacaacagattgattttattaccgaacaaaatgtttatgattatcggagtattactagtattggtaagggatacgactcaattactgaattgcacttcaatgcctttaatggtagtgccaaaggtacagaagtcttgattcaatcttctttagaagcagacaaggaagatatggctatcctctctctcctttcacgatactttcaaaatcgtggcattaagaaggtagattggctctataatgccaaccaagcagcgagtcgtggatatacctatcgtttggtggagattgcctttatcgataatgaacaagacatggcgatttttgaaaccaagaaagaggacattgcgaaaggtcttgtgtccgcaataacaggggttgaggtcaagacaattgtaccctcgacacccagttcaactgttgggagttcaggaactccttcaaaaccaatctatcttgttggtgatagtcttagggtgttgcctcatgcgactcattatcagactggtcagaaaatcgccaattgggtcaaggggcgcacctacaaaatcctccaagaaaaaaatgttcaccagtctaacagtttgagagcttatctacttgatggaatcaagtcatgggtgctgggcaggatgtagaaggaacaactaaaggccatagtgagcagacctatcaagcacagaaaggcgatacgtattatggtatcgctcggaagtttggtttaacagttgatgcccttcttgcggtaaatggcttgaagaagacggatattttaagagttggacaaactctaaaggtcaacgcagcttcaaggataacaaccgctattccaaccagtgttgcaagccgtgtggttgcgtcagcattatccaaggtcggtcaaaaggtaactgttccatctaacccttatggtggacagtgtgttgccttggtggataagattgttcaagaacttacggataagaatatgtcctatacaaatgccattgattgtttgaagaaagcaaaatcaaatggtttccaagtaatctatgatgcttggggtgtgaatcctaaagcaggtgatttctatgtcattgagacagatggtttggtctatgggcatattggtgtctgtgtgacggattctgatggaaaaagtattgatggtgtggaacagaatattgacggatattctgaccataataagaacggtatcaatgaccaattagaaattggtggaggtggaattactcgtcgtgtgaaacgtcaatggatggcggatggctcactctatgattctactggaacagttaaacttggaaaagttgtaggttggtttagaatttcataa

SEQ ID NO: 3 - DNA, 51 bp, Artificial Sequence
  forward primer
ggaattccatatgggaaaacatctagtcatttgtggtcatgggcaagggcg

SEQ ID NO: 4 - DNA, 60 bp, Artificial Sequence
  reverse primer
cgcggatccttaatgaaattctaaaccaaccaacaacttttccaagtttaactgttccag

SEQ ID NO: 5 - DNA, 48 bp, Artificial Sequence
  forward primer
gcatgccatgggaaaacatctagtgatttgtggacatgggcaaggacg

SEQ ID NO: 6 - DNA, 57 bp, Artificial Sequence
  reverse primer
gccgctcgagtgaaattctaaaccaacctacaacttttccaagtttaactgttccag

SEQ ID NO: 7 - DNA, 20 bp, Artificial Sequence
  forward primer
taatacgactcactataggg

SEQ ID NO: 8 - DNA, 19 bp, Artificial Sequence
  reverse primer
gctagttattgctcagcgg

SEQ ID NO: 9 - protein, 162 aa, Streptococcus dysgalactiae
GHGQGRTTYDPGAVNAKLGITEAGKVRELAKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFETKKEDIAKGLVSAIT

SEQ ID NO: 10 - protein, 45 aa, Streptococcus dysgalactiae
QTYQAQKGDTYYGIARKFGLTVDALLAVNGLKKTDILRVGQTLKV